(19) United States
(12) Patent Application Publication
(10) Pub. No.: US 2020/0372825 A1
(43) Pub. Date: Nov. 26, 2020

(54) INTELLIGENT GARMENT

(71) Applicant: Romina Ghassemi, Santa Monica, CA (US)

(72) Inventor: Romina Ghassemi, Santa Monica, CA (US)

(21) Appl. No.: 16/880,921

(22) Filed: May 21, 2020

Related U.S. Application Data
(60) Provisional application No. 62/850,863, filed on May 21, 2019.

(51) Int. Cl. G09B 19/00; A61B 5/11; A61F 5/02; A61F 5/01; A61B 5/00

(52) U.S. Cl. CPC G09B 19/003; A61B 5/1116; A61F 5/028; A61B 2560/0223; A61F 5/026; A61B 5/6804; A61B 2562/0219; A61F 5/0106

(57) ABSTRACT

The system provides an intelligent garment that includes sensors that can measure position, movement, acceleration, speed, distance, and the like. The sensors are coupled to a processing system that can interpret sensor data and provide real-time feedback and recommendations to the user (e.g. wearer of the garment). In one embodiment, the feedback may be aural via an earpiece or speaker. The system may include visual representations of desired movement or position via a device such as a smartphone, tablet, or other mobile device. The system can provide suggestions and corrections to the user during movement and/or activities, such as walking, sitting, golfing, tennis, throwing, dancing, and the like.

This patent application claims priority to U.S. Provisional Patent Application 62/850,863 filed on May 21, 2020, which is incorporated by reference herein in its entirety.

### BACKGROUND OF THE SYSTEM

There are many activities that can benefit from proper instruction, position, motion, motivation, teaching, and performance evaluation. Such is important for performance and for better health as well as reducing possible injury to relevant joints and supporting soft tissue. Even something as simple as sitting or walking, if done incorrectly, can lead to injuries, chronic pain, loss of movement or ability, loss of function, and the like. In the current art, the most common solution to activity evaluation involves a human coach in a live setting, or via live or pre-recorded video. A problem with such a system is that a coach or observer can be expensive. If not done live and in real-time, such coaching may allow bad habits to begin and persist.

In addition, it is difficult for an observer to be able to be fully aware of everything that is taking place during an activity, as the observer must focus on one or two things during each observation. An observer may miss important details, data, or other information that would be helpful to instruct or correct the person performing the activity.

### SUMMARY

The system provides an intelligent garment that is adjustable to provide customizable support to correct and improve posture. The garment also includes sensors which measure user biometrics, position, movement, acceleration, speed, distance, and the like. The sensors are coupled to a processing system that can interpret sensor data and provide real-time feedback and recommendations to the user (e.g. wearer of the garment). In one embodiment, the feedback may be aural via an earpiece or speaker. The system may include visual representations of desired movement or position via a device such as a smartphone, tablet, or other mobile device. The system can provide suggestions and corrections to the user during any body range of motion movements, such as walking, sitting, golfing, tennis, throwing, dancing, and the like. The instructions can be specific as to which body part needs correction and the best manner in which to do so. The sensors can provide data through wired or wireless connection to a processing system, such as a smartphone, which then can compare the movement to a baseline and/or target movement, calculate error from the desired movement, generate an appropriate command, and then present the command to the user, either audibly and/or visually. The system can also record movements so that they can be replayed later as desired. In one embodiment, the system can also suggest manually adjusting biomechanically positioned integrated bands and straps in the garment to improve position, and/or includes methods of automatically adjusting the size and/or shape of portions of the garment to improve user position and performance.

### BRIEF DESCRIPTION OF THE DRAWINGS

FIGS. 1A and 1B are an example of the front and back respectively of an intelligent garment in an embodiment of the system.

FIG. 2 is a flow diagram illustrating target posture in an embodiment of the system.

FIG. 3 illustrates an initial posture of a user in an embodiment of the system.

FIG. 4 illustrates a target posture in an embodiment of the system.

FIG. 5 is a flow diagram illustrating sensor calibration in an embodiment of the system.

FIG. 6 is a flow diagram illustrating activity coaching in an embodiment of the system.

FIG. 7A is a view of a leg of a garment in an embodiment of the system.

FIG. 7B is a view of a pant in an embodiment of the system.

FIG. 8 is a perspective view of a garment with a side zipper in an embodiment of the system.

FIG. 9 illustrates a rear view of a Thoracic Lumbar Sacral Orthosis (TLSO) brace in an embodiment of the system.

FIG. 10 illustrates a front view of a TLSO brace in an embodiment of the system.

FIG. 11 illustrates a knee attachment in an embodiment of the system.

FIG. 12 illustrates use of the system in an embodiment.

FIG. 13 illustrates the System App functions in an embodiment of the system.

FIG. 14 illustrates an example processing environment in an embodiment of the system.

### DETAILED DESCRIPTION OF THE SYSTEM

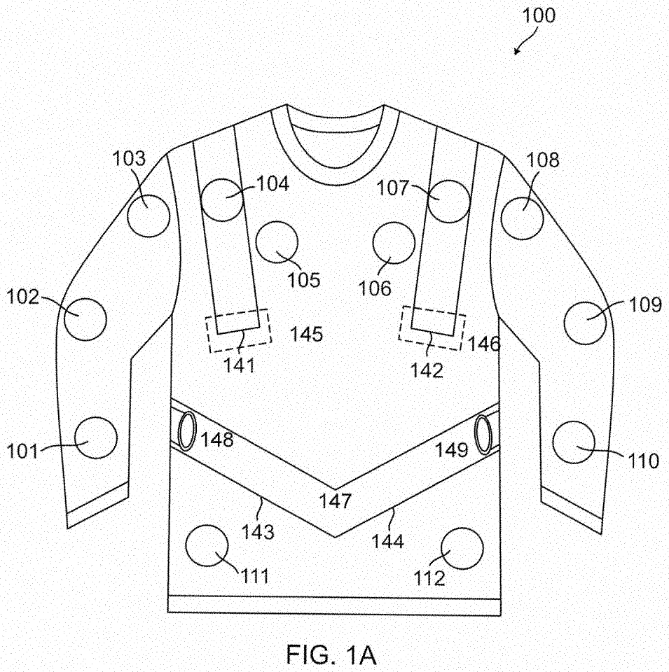

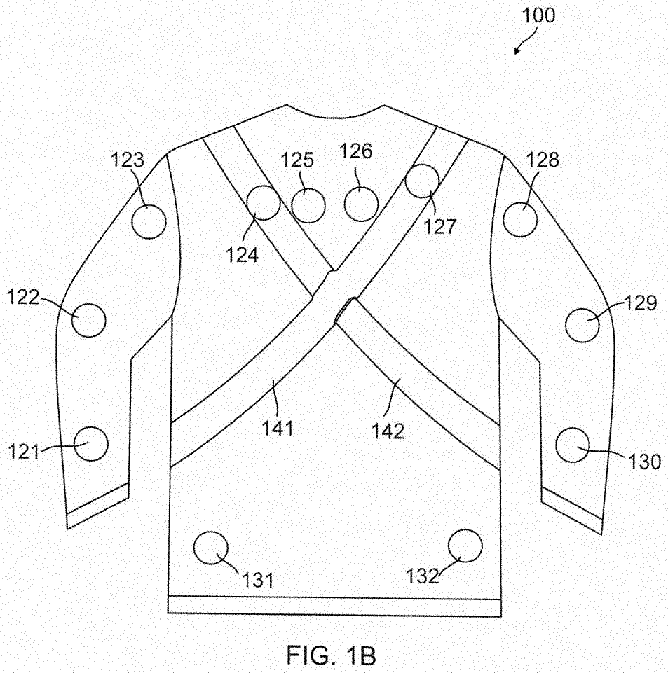

The system provides an intelligent garment coupled with a processing and analysis system to enable a user to improve posture and body alignment, performance, and the like when wearing the garment. FIG. 1 is an example of an intelligent garment in an embodiment of the system. The example of FIGS. 1A and 1B is of a long-sleeved shirt, but the system has equal application to short sleeve shirts, pants, individual leggings, sports bra, one-piece body suits, individual sleeves, gloves, hats, headbands, neck bands, stockings, shoes, shoe inserts, and the like.

Shirt

The garments of the system include mechanisms to provide posture and other support. The garments include shirts, pants, braces, vests, and the like. On embodiment of a shirt is shown in FIG. 1. The shirt 100 is illustrated from a front view in FIG. 1A and a rear view in FIG. 1B. Shirt 100 includes adjusting straps 141 and 142 coming from the back of the shirt over the shoulder to the front of the shirt. In one embodiment the straps can extend to approximately mid chest of the shirt, at attachment points 145 and 146. In one embodiment, attachment points 145 and 146 are positioned higher on the shirt, near sensor locations 104 and 107. Straps 141 and 142 may be visible or may be partially hidden in channels defined in the garment to receive the straps. When hidden in the garment, the ends of straps 141 and 142 protrude from the end of the channels so that they can be gripped by the user and pulled to the attachment points 145 and 146 to adjust the fit of the garment.

In one embodiment, the ends of straps 141 and 142 include Velcro® that can engage corresponding Velcro® at attachment points 145 and 146. The attachment points are wide enough to allow a range of placement of the ends of straps 141 and 142 to provide custom support to the user.

Referring now to the rear view of FIG. 1B and the front view of FIG. 1A, it can be seen that adjusting straps 141 and 142 go over the shoulder to cross over to the opposite waist/abdomen region. Strap 141 leaves the chest over the right shoulder and crosses diagonally across the back and around the waist to ring 149 on the left side of the waist. Strap 142 goes over the left shoulder and crosses diagonally across the back and around the waist ending at ring 148 on the right side of the waist. The rings 148 and 149 are used to pull the strap toward region 147 for attachment. The rings 148 and 149 may instead be the end of straps 141 and 142 with no ring present. Region 147 is V-shaped in one embodiment but may be X-shaped as well. The ends of straps 141 and 142 near rings 148 and 149, and region 147, may include Velcro® to allow the straps to be secured at a plurality of locations for a custom fit for the user.

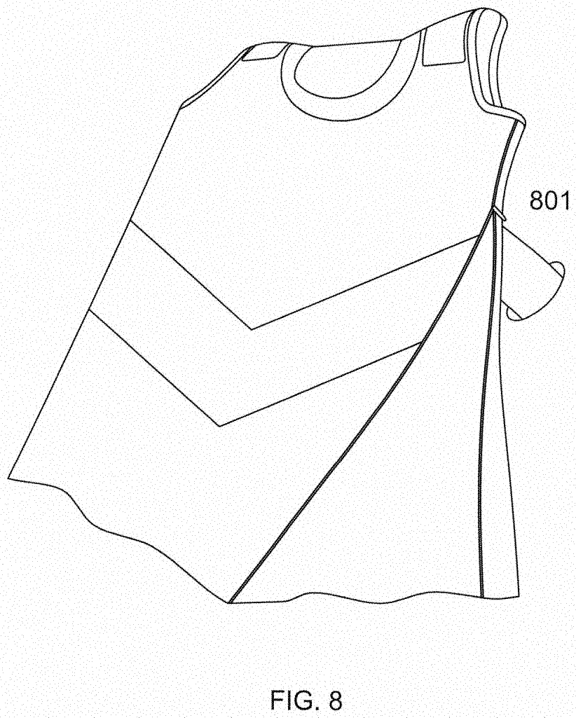

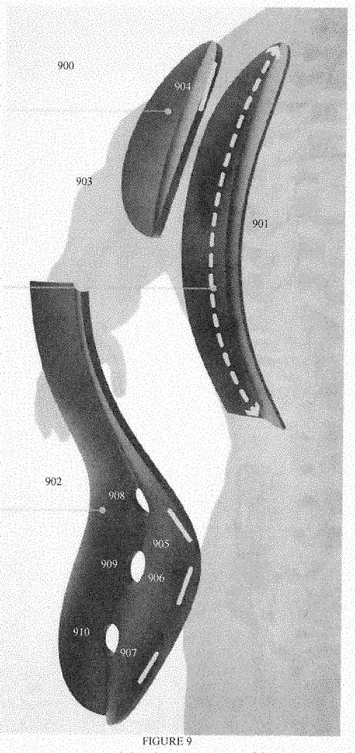

The straps allow a user to lock their shoulders at a desired position. In one embodiment, the straps reposition the alignment of the shoulders. The straps allow the user to adjust their shoulders in an optimal backward position. It has been found that this helps provide improved range of motion and performance in many activities, including sports, dance, walking, running, sitting, and the like. FIG. 8 illustrates an embodiment of the garment with a zipper 801 on the side that can extend all the way up on side of the garment to make it easier to put on and remove, as well as another form of adjustment for a user when wearing the garment. This embodiment may be used with or without the sensors.

Brace

There are some back conditions that require pressure to be put on certain parts of the spine. One typical mechanism for applying the pressure is a Thoracic Lumbar Sacral Orthosis (TLSO) brace that supports the thoracic, lumbar, and sacrum part of the spine. A typical TLSO brace has pads in the front and back as well as shoulder supports to keep the pads in place.

The brace of the present system can be integrated into the shirt of FIGS. 1A and 1B or be used on its own. The brace can stabilize the spine for better posture. The straps of the brace described below work as alignment tension straps to reduce inter-discal space. The brace 900 comprises a rear portion and a front portion. The rear portion is illustrated in FIG. 9. The brace 900 in one embodiment comprises a neck region 901, base region 902, and shoulder strap panel 903. The brace 900 extends in one embodiment from T2 (thoracic vertebrae number 2) to S2 (Sacral vertebrae number 2).

The neck region 901 can be slid into a slotted portion of base 902 and be adjustable in height to allow the brace to properly fit a variety of users. The shoulder strap panel 903 includes openings 904 to receive crossing straps, such as straps 141 and 142 of FIG. 1. These straps wrap around the user's shoulder and waist to help hold the strap panel 904 and correspondingly, the brace 900, in place during use. The strap panel 904 can also be slid into a slot in base member 902 in an embodiment.

The base region 902 includes slots 905, 906, and 907 (repeated on the other side of 902) that receive elastic straps (not shown) that can be wrapped around the torso of the user and connected at a front pad to retain the base region in place and against the correct portion of the spine during use. The straps are pulled tight to fit and fastened in the front of the user at an abdominal pad.

Base region 902 includes openings 908, 909, and 910. In one embodiment, the elastic straps are coupled to a small block that fits into the opening and helps apply pressure to separate certain vertebrae to remove stress on the back. In one embodiment, the system comprises only top region 901 and bottom region 902, with strap openings provided in top region 901 to receive the straps such as straps 141 and 142.

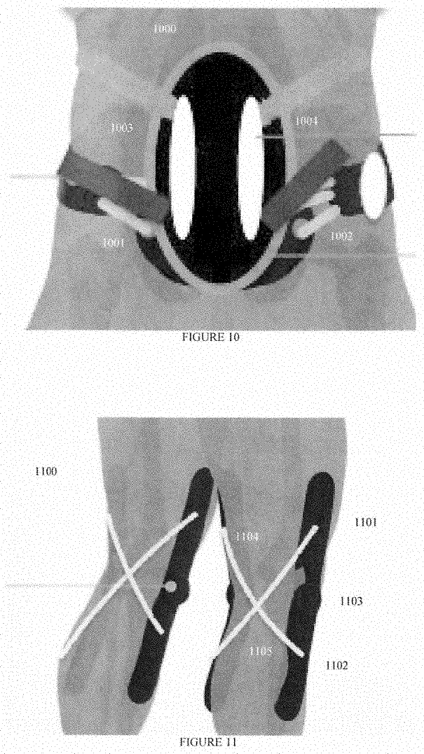

FIG. 10 illustrates the front pad of the brace of FIG. 9. The front pad 1000 is approximately oval shaped. The pad 1000 includes attachment regions 1001 and 1002 to receive the adjusting straps from the rear panel. The attachment regions may be comprised of Velcro® or some other means of securing and stabilizing the straps. Regions 1003 and 1004 can receive and secure straps from the upper portion of the back brace.

Pant

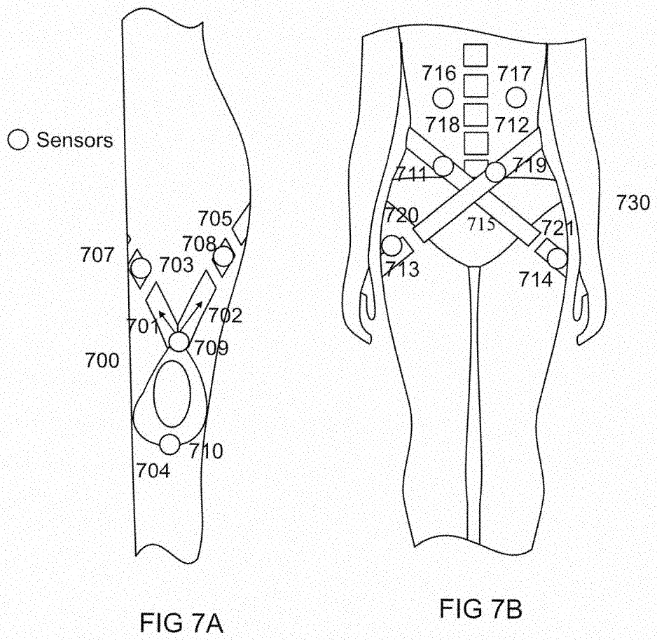

FIG. 7A illustrates an embodiment of the system in a pant. FIG. 7A illustrates straps 701 and 702 built into a garment 700 in the area of the knee. The straps extend from a horseshoe pad 704 built into the garment 700 at the knee region. The elastic tension straps stabilize above and below the patella and are adjusted in place by straps 701 originating from behind the knee and securing in front above right and front left 702 via Velcro that can engage Velcro region 703 and 705 on the garment 700. In one embodiment, the garment 700 includes sensors 707, 708, 709, and 710 that can be used with the coaching system to provide feedback during activities (such as walking and running). Other sensor locations and additional sensors may also be used.

FIG. 11 illustrates a knee support structure in an embodiment of the system. The knee support 1100 includes a rigid upper leg 1101 and rigid lower leg 1102 joined by a ratcheted dial 1103. The legs 1101 and 1102 can be applied to the inside and outside of the knee on one or both legs. The legs are secured in place by elastic tension bands 1104 and 1150. The angle of the legs 1101 and 1102 can be set by the ratcheted dial 103. In one embodiment the legs can be set at angles to each other of 15, 45, 90, and 135 degrees to provide customized knee joint support. This system allows the user to have customized knee joint protection. This assembly can be integrated into the garment as desired or applied externally.

FIG. 7B illustrates an embodiment of the system in a garment 730. Straps 711 and 712 have one type of Velcro fastener at the ends and are mounted on the front of the garment 730 and wrap around and criss-cross to engage the other type of Velcro regions 713 and 714, respectively. This embodiment is used to provide Sacrum stabilization at region 715. As noted above, the straps can be integrated with the garment in built in channels or not as desired. The garment 710 can include sensors 716, 717, 718, 719, 720, and 721 in one embodiment. Additional sensor locations and additional sensors may also be used.

The system can also include sensors in a hat, headband, earphone, ear buds, and the like, to help determine head position during activities. In addition, the system can include sensors in shoes and gloves so that feet and hand position can be determined.

Activity Feedback and Training System

In addition to correcting, optimizing/changing posture, the garment of the present system helps track performance, provide tips on positioning, and can teach a user improved performance technique. The garment includes sensors at strategic locations which measure user biometrics, position, movement, acceleration, speed, distance, and the like. The sensors are coupled to a processing system that can interpret sensor data and provide real-time feedback and recommendations to the user (e.g. wearer of the garment).

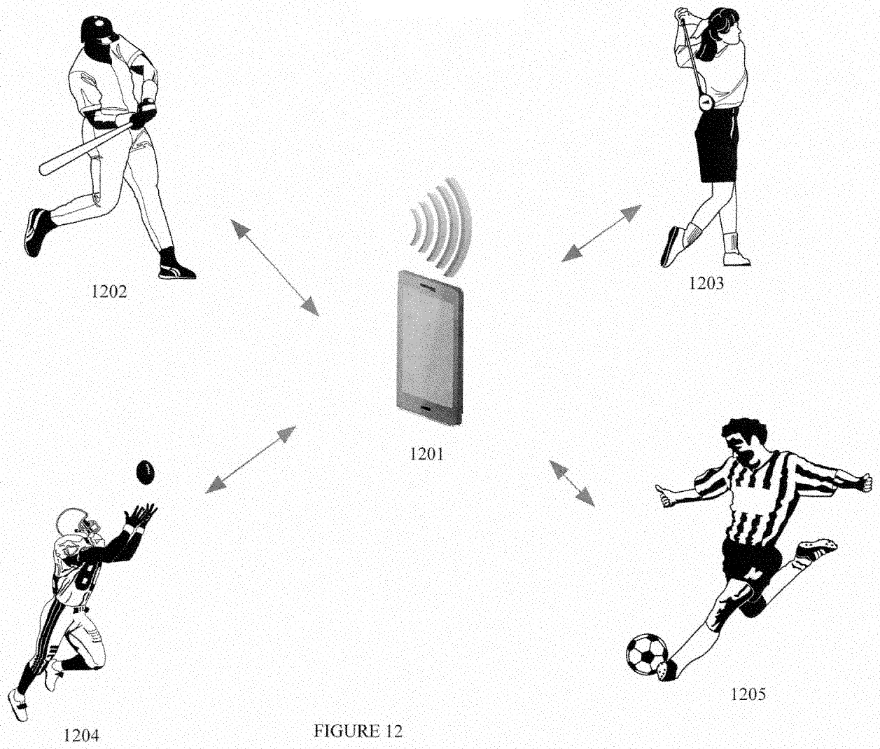

FIG. 12 illustrates example applications of the system in an embodiment. The system includes a System App implemented on smartphone 1201. As noted, the smartphone may be any suitable processing device, including laptops, tablets, smart glasses, smart watches, and the like. The system may be used to help train a user in a variety of general ranges of motion and muscle training activities, including, but not limited to, recreational activities such as baseball 1202, golf 1203, football 1204, soccer 1205, running, walking, and the like.

Smartphone 1201 receives signals from sensors embedded in the garment worn by the user. The smartphone 1201 processes the signals and determines the status of the user in performing the activity. The System App compares the performance to a goal performance and identifies corrections and/or tips to be suggested to the user. The System App then can communicate the suggestions to the user in a number of ways. For example, the user may receive audio cues through headphones (wired or wireless). Instead of, or in addition to, the audio suggestions, the smartphone 1201 may display images that show what the user is doing incorrectly and present a target performance.

Consider the golfer 1203. The sensors in the garment allow the smartphone to determine the stance of the golfer when preparing to strike the ball. The System App can provide audio information concerning the posture, arm and leg position, and the like. Then, during the swing, the system can locate the position of the user's body during the swing and provide immediate feedback by showing the user's swing overlaid on a target swing, with corrective suggestions presented to the user. Each swing can be stored in the smartphone and replayed later as desired.

In one embodiment, the feedback may be aural via an earpiece or speaker. The system may also include visual representations of desired movement or position via a device such as a smartphone, tablet, or other mobile device. The system can provide suggestions and corrections to the user during an activity, such as walking, sitting, golfing, tennis, throwing, dancing, and the like. The instructions can be specific as to which body part needs correction and the best manner in which to do so. The sensors can provide data through wired or wireless connection to a processing system, such as a smartphone, which then can compare the movement to a baseline and/or target movement, calculate error from the desired movement, generate an appropriate command for correction of the error, and then present the command to the user, audibly and/or visually.

The system can also record movements of the user during the activity so that the movements can be replayed later as desired. In one embodiment, the system can also suggest manually adjusting biomechanically positioned integrated bands and straps in the garment to improve posture and/or position, and/or includes methods of automatically adjusting the size and/or shape of portions of the garment to improve user position and performance. In one embodiment, the sensors can provide biometric data about the user that can be used for medical analysis, health, and wellness.

Sensors

Referring again to FIG. 1A, the shirt 100 includes a plurality of sensors 101-112 embedded in the fabric of the shirt 100. The sensors may be placed in pockets of the garment on the inside or outside, and in one embodiment are removable for each washing. In one embodiment the sensors can be weaved into the fabric for ease of care for a user.

Sensors 101 and 110 are near the wrist or forearm of the shirt, with sensors 102 and 109 near the elbow. Sensors 103 and 108 are near the shoulder joint of the user. Sensors 104 and 107 are on the upper chest near the shoulder, while sensors 105 and 106 are lower and near the middle of the chest of shirt 100. Sensors 111 and 112 are near the waist of the shirt 100.

FIG. 1B shows the back side of shirt 100. The shirt 100 includes sensors 121 and 130 at the forearm/wrist area, sensors 122 and 129 at the elbow area, and sensors 123 and 128 in the upper arm/shoulder region. Sensors 124 and 126 are in the upper back near the deltoid region and sensors 125 and 127 are near the trapezius region. Sensors 131 and 132 are near the lower back region.

It should be noted that the system can operate with more or fewer sensors as desired, depending on the activity being done by the user. In addition, the sensors may be located in different locations as appropriate, without departing from the scope or spirit of the system.

In one embodiment, the sensors are battery powered and can be turned on and off by an application on a smartphone or other mobile device. In one embodiment, the sensors can be turned on and off manually. In one embodiment the sensors 101-112 each have a unique digital identification and a unique physical identification on the sensor and are intended to be placed back in the same location after removal. In one embodiment, the sensors 101-112 have a unique digital identification and can be placed in any location after removal. In this embodiment, a calibration set-up procedure is run to identify which sensor is in which location so that they can be mapped to the correct location for the analysis software.

The sensors should be able to provide their own identification information, location, and status during an initial phase. During use, the sensors should provide acceleration information, position information, gyroscopic information, deflection information, relative position to other sensor information, gait analysis, cadence measurements, load fatigue, effort, stress, QRS, biometric information, surface EMG, muscle activity reaction, and the like. The system can use this information to provide performance analysis to the user, along with health analysis and recommendations.

In one embodiment, the sensors can also detect the user pulse, temperature, oxygenation, respiration, blood sugar level, EKG, EEG, heart rate recovery, and the like. The garment can be used as part of a telemedicine environment where the sensors provide information about the user to medical professionals. The garment can be used with medical therapies, physical therapy, occupational therapy, therapeutic exercise or activity, gait training, physiologic measurements, neuromuscular re-education (e.g. after a stroke or neurological event), use with prosthetic limb, and the like.

The sensors are rechargeable to allow for re-use. An example of sensors that can be used in an embodiment of the system include Hexoskin health sensors, Spire health monitors, ACI system sensors, mbientlab wireless environmental sensors, electrical, textile, tactile, piezoelectric, pressure and/or Nano-sensor technology, and the like. In one embodiment, the sensors have rechargeable and/or replaceable batteries. In one embodiment the sensors can be coupled to a wire harness embedded in the garment so that the sensors can be hardwired to a processing device. In one embodiment the sensors can be recharged wirelessly and/or via a USB or other suitable connection.

Sensor Calibration

FIG. 2 is a flow diagram illustrating the calibration of the sensors in an embodiment of the system. The purpose of calibration is to determine the position of each sensor, the relative position of each sensor to other sensors, and to determine the operational readiness of the sensors to perform as desired.

At step 201 the user puts the garment on and initiates the calibration sequence via the smartphone. The calibration sequence is presented to the user as a series of instructions and/or graphical cues on the display of the smartphone via the System App. In one embodiment, the user may be presented with a graphical image to identify the sensors and garments being used. For example, the user might only be wearing a shirt, and so the system will not look for sensors in pants, shoes, gloves, hats, or earbuds. In addition, the user might have a short sleeve shirt instead of a long sleeve shirt, affecting the number of sensors that are in use. In addition, the user may have decided to not use all possible sensors in the garment. By identifying which garment and sensors are being used, the calibration sequence can be more efficient. In one embodiment, instead of the user identifying the sensors and garments, the system can present the user with a series of questions to help identify the configuration.

At step 202 the system pings each of the sensors to confirm the presence of the sensor and its operational status. If there are any issues the system may suggest corrective measures, such as battery charging, sensor replacement, reboot or reset of the sensors, and the like.

At step 203 the system presents a movement on the smartphone that the user is to execute. Examples of a movement that might be presented include lifting right arm, lifting left arm, torso twisting, bending, jumping jacks, arm swings in a horizontal and/or vertical plane, and the like. The display of the smartphone may display a graphical representation of each desired movement. In one embodiment, the user attempts to synchronize their movement with the movement on the display, which can aid in the calibration sequence.

Other movements may include lifting the arm in the Sagittal plane, lifting the right arm, lifting the left arm to shoulder level and next to ear within comfort to side of body, and lifting the arm in a horizontal plane across the body at shoulder level. Movement can include raising the right arm to shoulder level and cross the chest towards the left shoulder, and raising the left arm to shoulder level and cross the chest towards the right shoulder, Another movement may be lifting the left arm to shoulder level and to face level in comfort. One embodiment may include standing in a neutral position and rotating the torso twisting right within a comfort zone, back to neutral and rotating the torso twisting within comfort to the left. The user may be standing in a neutral position bending forward or standing neutral and bending backwards. The user may be instructed to lift arms to shoulder level, flex palms, and push forwards. The user may be standing in a neutral position with arms to the side and rotate right and left and/or with palms outward to optimal comfort.

In one embodiment, the system requests the user to go through the Range of Joint Motion Evaluation Chart, such as provided by the Washington State Department of Social Services at https://www.dshs.wa.gov/SiteS/default/files/FSA/forms/pdf/13-585a.pdf of incorporated by reference herein

At step 204 the system receives data from the sensors which it uses to calibrate the sensors. For example, since each sensor has a unique ID, the system can determine the position of the sensors based on the calibration motions. This is particularly useful when the sensors may be placed in any of the pouches. The system can detect the sensors that are presently moving and identify in which pouch of the garment the sensors are placed. At step 205 the system indicates calibration based on that movement. This may be a visual indicator, audible, vibration or the like. In one embodiment, one or more sensors are treated as a baseline sensor and the relative distance between that baseline sensor and each of the sensors being calibrated is used to provide position information and other information necessary to calibrate the sensors.

At decision block 206 the system determines if there are additional calibration movements to perform. If so, the system returns to step 203 and presents the next calibration movement to the user. If not, the system proceeds to step 207 and indicates completion. At this point the system processing has located and calibrated the sensors and has normalized any discrepancies in actual sensor performance with ideal sensor performance.

Target Posture

In one embodiment, the system helps generate a baseline status of the user so to determine the amount of correction and/or teaching required, as well as to be able to provide progress analysis from this baseline set of conditions.

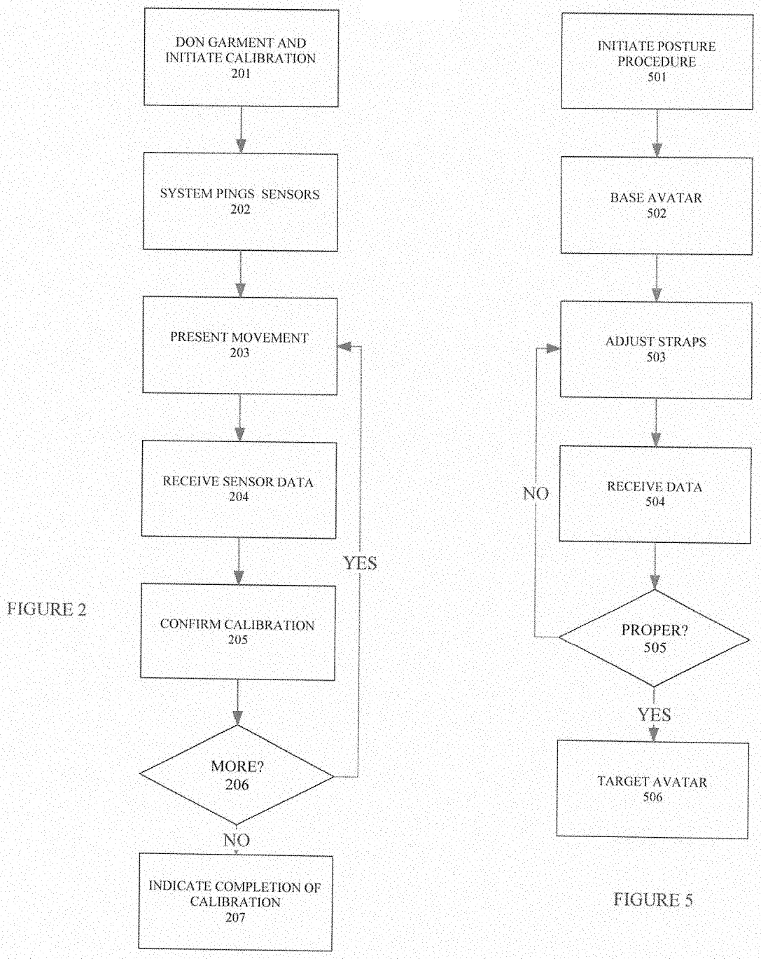

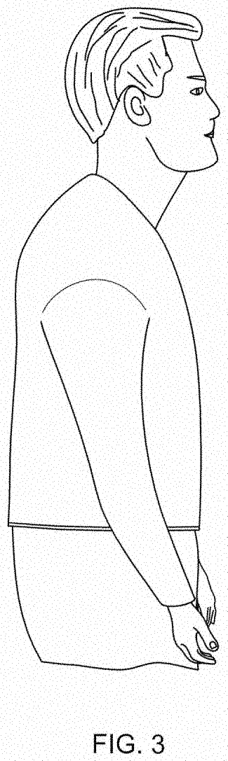

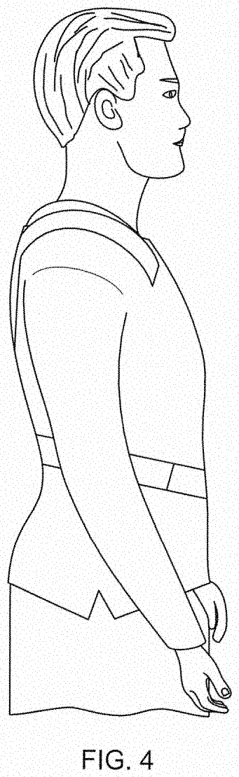

FIG. 5 is a flow diagram illustrating the operation of the system in an initial phase in one embodiment. At step 501 the user activates the posture procedure using the processing device. For purposes of example, we will refer to the processing device as a smartphone, but it is understood that any processing device may be utilized, including tablet devices, laptops, mobile processing devices, and the like.

At step 502 the user creates a base avatar displaying a base posture condition for use with the system by standing in a natural rest position (e.g. as shown in FIG. 3). The sensors are polled for their relative positions and the base avatar is graphically displayed on the smartphone and stored in the system. The system then attempts to change the posture of the user to a target posture, and thereby create a target avatar for use in the training of the user.

At step 503 the user adjusts the straps of the garment in response to instructions from the System App. The user may be instructed to adjust one or both of the shoulder straps and/or one or both of the waist straps. In one embodiment, the attachment regions may have identifiable lines (e.g. numbered, lettered, qualitative, and the like) and the user may be instructed to pull a strap to a designated location on the attachment region. For example, the user may be instructed to pull the left shoulder strap to a second position on the attachment region, and to pull the right strap to a third position on the attachment region, depending on the posture of the user.

At step 504 the system polls and receives data from the repositioned sensors and determines if the user is in a proper posture at decision block 505. If not, the system returns to step 503 and the user re-adjusts the straps.

If the user does have correct posture at decision block 505, the system proceeds to step 506 and defines this state as the target avatar as shown in FIG. 4. The target avatar is used in the future as a baseline for the user before beginning activities. In one embodiment, the user may repeat the movements of FIG. 2 to recalibrate the sensors with the new posture.

In one embodiment, the garment may be used without the sensors but using the straps to adjust posture from initial posture to target posture. The user can adjust straps 141 and 142 accordingly to aid in attaining and maintaining the target posture when wearing the garment.

Activity Coaching

After the user has calibrated the sensors and adjusted the posture straps, the user is ready to begin an activity. This is where the system enables real time training and coaching for the user. The system includes the ability to produce audible speech via pre-recorded messages, text to speech, or via some other mode. During an activity, the system monitors the sensors and provides audio feedback to the user via wired or wireless earphones, headphones, ear buds, or the like. The data from the sensors is analyzed and an appropriate audible communication is triggered in response to sensor data.

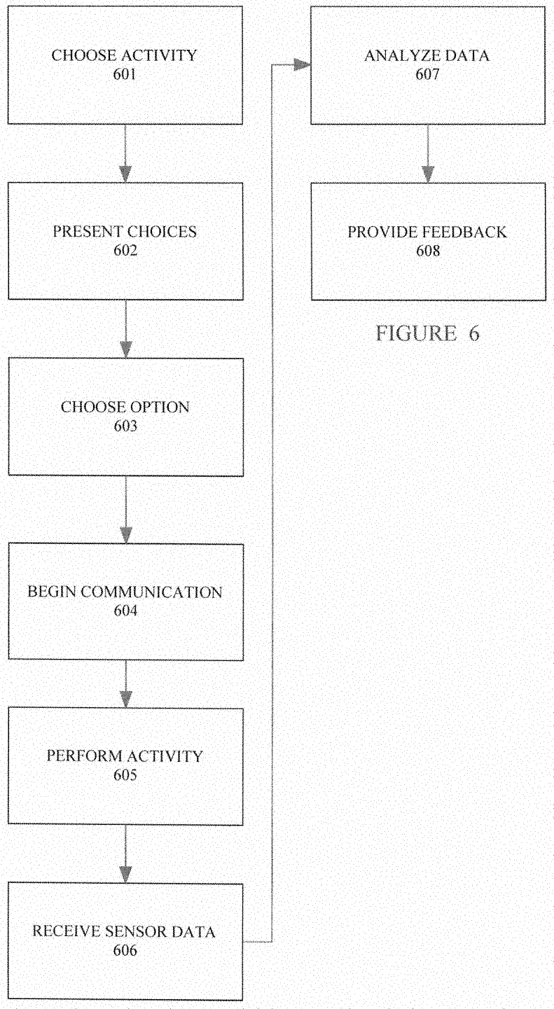

FIG. 6 is a flow diagram illustrating the coaching of the system in an embodiment of the system. At step 601 the user chooses the activity to be performed and active ranges of motion, (e.g. throwing/shooting, (football/basketball), swinging, (golfing), batting, pitching, (baseball), shoulder swings, (tennis), kicking (soccer), weightlifting, volleyball, and the like.) At step 602 the system presents a plurality of choices to the user of coachable aspects of the activity. For example, consider a user who wants to practice golfing. The system may present a choice of which club is being used, as well as which part of the activity to work on. For example, the user may select stance, swing, follow-through, and the like. In one embodiment, the system combines an entire movement into an activity, with, for example, stance, swing, and follow-through done continuously, with feedback on all three aspects being provided after the activity.

At step 603 the user selects the options presented at step 602 and begins the activity. At step 604, the system may begin communicating with the user with reminders on setup, stance, posture, and the like.

At step 605 the user performs some or all of the activity. At step 606 the system receives data from the sensors. At step 607 the system analyses the data to determine the actual performance of the user as compared to a target standard of performance. The target standard may be an intermediate stage between novice and expert, or it may represent a desired end state with no intermediate states. At step 608 the system provides feedback to the user. The feedback may be audio in the user's earphones, and/or it may include a visual reproduction of the activity based on sensor movement overlaid with a target motion, allowing the user to see where the differences are. The system can provide coaching and feedback on how to correct deficiencies in the performance. In one embodiment, the user can touch various sensor points or areas on the simulation of the activity and receive tips and coaching on how to improve that particular portion. The user may also pause the playback at any moment and receive coaching and feedback on that portion of the activity.

An advantage of the system is that it can provide coaching for both static moments and dynamic motion of the activity, along with initial start point and end point, improving the user in all aspects of the activity. The system can suggest exercises that can be done with or without the system being engaged as the user desires. In some cases, the exercises are not the activity itself, but are exercises that can improve the user performance when performing the activity. In one embodiment, even the exercises can be monitored via the smart garment and sensors so that the user is always using optimum technique to achieve desired results.

In addition to the simulated motion based on the sensors, the system can provide video examples of proper or desired technique to the user. The simulated motion can be overlaid with the video so the user can see where differences are and attempt to correct them.

System App

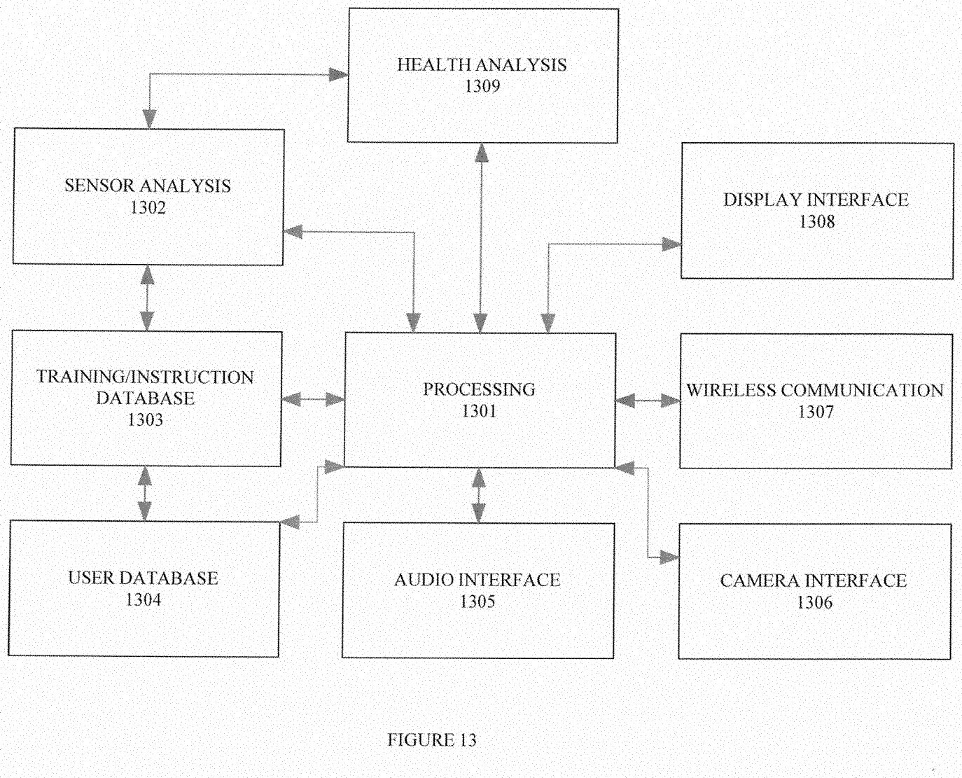

The System App is illustrated in an embodiment in FIG. 13. The System App includes processing module 1301 that interfaces and communicates with all of the other modules. The Sensor Analysis Module 1302 receives sensor information that is provided via the Wireless Communication Module 1307 and sent via Processing Module to the Sensor Analysis Module 1302. The Sensor Analysis Module 11302 interprets the sensor data to generate position, movement, location, and other information related to the activity.

The Sensor Analysis Module 1302 provides the activity data to Training/Instruction Database 1303 which generates instructions, corrections, suggestions, and the like based on the activity data. The Training/Instruction Database 1303 collects user data from the User Database 1304, which includes the user baseline avatar information, activity targets, progress information, and the like.

When the System App has generated instructions for the user from the Training/Instruction Database 1303, they are sent via the Processing Module 1301 to the Audio Interface 1305 and/or the Display Interface 1308 to present to the user.

The Camera Interface 1306 may also be part of the system and the user may video the activity with the Camera Interface recording the images and correlating the images with sensor data to provide more accurate training.

The Health Analysis Module 1309 can collect health related information provided by the sensors and provide warnings to the user of any detected health issues related to the activity and or other conditions.

### Example Computer Environment

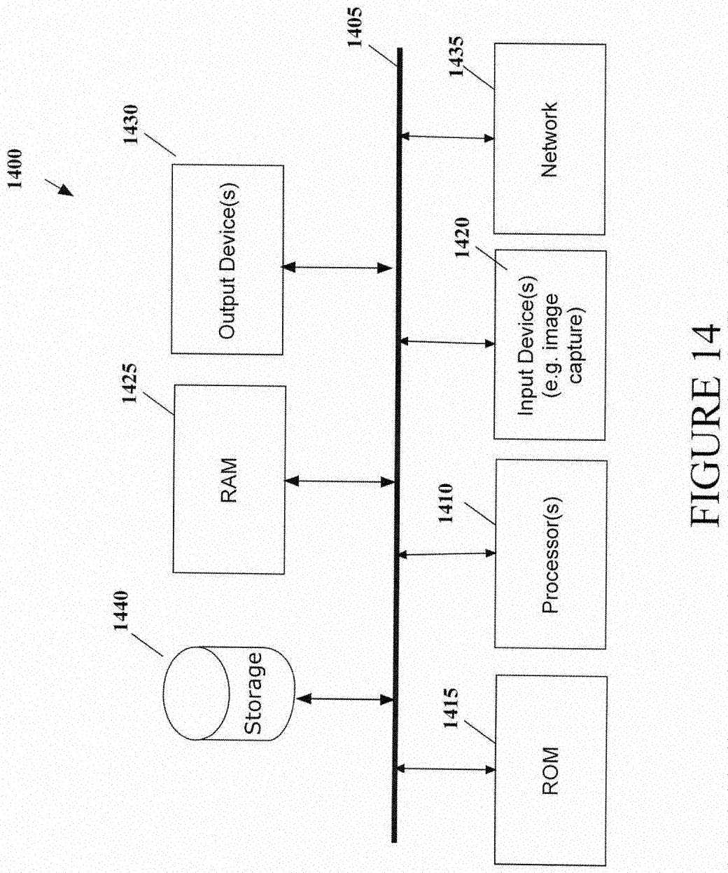

FIG. 14 illustrates an exemplary a system 1400 that may implement the system. The electronic system 1400 of some embodiments may be a mobile apparatus. The electronic system includes various types of machine-readable media and interfaces. The electronic system includes a bus 1405, processor(s) 1410, read only memory (ROM) 1415, input device(s) 1420, random access memory (RAM) 1425, output device(s) 1430, a network component 1435, and a permanent storage device 1440.

The bus 1405 communicatively connects the internal devices and/or components of the electronic system. For instance, the bus 1405 communicatively connects the processor(s) 1410 with the ROM 1415, the RAM 1425, and the permanent storage 1440. The processor(s) 1410 retrieve instructions from the memory units to execute processes of the invention.

The processor(s) 1410 may be implemented with one or more general-purpose and/or special-purpose processors. Examples include microprocessors, microcontrollers, DSP processors, and other circuitry that can execute software. Alternatively, or in addition to the one or more general-purpose and/or special-purpose processors, the processor may be implemented with dedicated hardware such as, by way of example, one or more FPGAs (Field Programmable Gate Array), PLDs (Programmable Logic Device), controllers, state machines, gated logic, discrete hardware components, or any other suitable circuitry, or any combination of circuits.

Many of the above-described features and applications are implemented as software processes of a computer programming product. The processes are specified as a set of instructions recorded on a machine-readable storage medium (also referred to as machine readable medium). When these instructions are executed by one or more of the processor(s) 1410, they cause the processor(s) 1410 to perform the actions indicated in the instructions.

Furthermore, software shall be construed broadly to mean instructions, data, or any combination thereof, whether referred to as software, firmware, middleware, microcode, hardware description language, or otherwise. The software may be stored or transmitted over as one or more instructions or code on a machine-readable medium. Machine-readable media include both computer storage media and communication media including any medium that facilitates transfer of a computer program from one place to another. A storage medium may be any available medium that can be accessed by the processor(s) 1410. By way of example, and not limitation, such machine-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a processor. Also, any connection is properly termed a machine-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc where disks usually reproduce data magnetically, while discs reproduce data optically with lasers. Thus, in some aspects machine-readable media may comprise non-transitory machine-readable media (e.g., tangible media). In addition, for other aspects machine-readable media may comprise transitory machine-readable media (e.g., a signal). Combinations of the above should also be included within the scope of machine-readable media.

Also, in some embodiments, multiple software inventions can be implemented as sub-parts of a larger program while remaining distinct software inventions. In some embodiments, multiple software inventions can also be implemented as separate programs. Any combination of separate programs that together implement a software invention described here is within the scope of the invention. In some embodiments, the software programs, when installed to operate on one or more electronic systems 1400, define one or more specific machine implementations that execute and perform the operations of the software programs.

The ROM 1415 stores static instructions needed by the processor(s) 1410 and other components of the electronic system. The ROM may store the instructions necessary for the processor(s) 1410 to execute the processes provided by the system. The permanent storage 1440 is a non-volatile memory that stores instructions and data when the electronic system 1400 is on or off. The permanent storage 1440 is a read/write memory device, such as a hard disk or a flash drive. Storage media may be any available media that can be accessed by a computer. By way of example, the ROM could also be EEPROM, CD-ROM or other optical disk storage, magnetic disk storage or other magnetic storage devices, or any other medium that can be used to carry or store desired program code in the form of instructions or data structures and that can be accessed by a computer.

The RAM 1425 is a volatile read/write memory. The RAM 1425 stores instructions needed by the processor(s) 1410 at runtime, the RAM 1425 may also store the real-time video or still images acquired by the system. The bus 1405 also connects input and output devices 1420 and 1430. The input devices enable the user to communicate information and select commands to the electronic system. The input devices 1420 may be a keypad, image capture apparatus, or a touch screen display capable of receiving touch interactions. The output device(s) 1430 display images generated by the electronic system. The output devices may include printers or display devices such as monitors.

The bus 1405 also couples the electronic system to a network 1435. The electronic system may be part of a local area network (LAN), a wide area network (WAN), the Internet, or an Intranet by using a network interface. The electronic system may also be a mobile apparatus that is connected to a mobile data network supplied by a wireless carrier. Such networks may include 3G, HSPA, EVDO, and/or LTE.

It is understood that the specific order or hierarchy of steps in the processes disclosed is an illustration of exemplary approaches. Based upon design preferences, it is understood that the specific order or hierarchy of steps in the processes may be rearranged. Further, some steps may be combined or omitted. The accompanying method claims present elements of the various steps in a sample order and are not meant to be limited to the specific order or hierarchy presented.

The various aspects of this disclosure are provided to enable one of ordinary skill in the art to practice the present invention. Various modifications to exemplary embodiments presented throughout this disclosure will be readily apparent to those skilled in the art, and the concepts disclosed herein may be extended to other apparatuses, devices, or processes. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 18(f) unless the element is expressly recited using the phrase “means for” or, in the case of a method claim, the element is recited using the phrase “step for.”

Thus, an intelligent garment has been described.

What is claimed is:

1. A garment comprising:
a plurality of sensors disposed in the garment for communicating to a processing device;
a posture adjusting mechanism integrated into the garment for adjusting posture of a user of the garment.

2. The garment of claim 1 wherein the sensors communicate wirelessly with the processing device.

3. The garment of claim 2 wherein the processing device is a smartphone.

4. The garment of claim 3 wherein the sensors detect one or more of muscle reaction, tension, position, rotation, movement, and acceleration.

5. The garment of claim 4 wherein the posture adjusting mechanism comprises first and second straps that can be secured at a plurality of positions.

6. The garment of claim 5 wherein the smartphone can analyze sensor data and provide training to the user.

7. The garment of claim 6 wherein the garment is a shirt.

8. The garment of claim 6 wherein the garment is a pant.

9. The garment of claim 7 further including a brace.
